(19) United States
(12) Patent Application Publication
(10) Pub. No.: US 2021/0030380 A1
(43) Pub. Date: Feb. 4, 2021

(54) SYSTEM AND METHOD TO VISUALIZE BOTH SOFT-TISSUE AND HARD BONE ANATOMY OF AN OBJECT

(71) Applicant: PANACEA MEDICAL TECHNOLOGIES PRIVATE LIMITED, Banagalore (IN)

(72) Inventors: Goteti Venkata SUBRAHMANYAM, Banagalore (IN); Avinash Rao KUPPA, Banagalore (IN)

(73) Assignee: Panacea Medical Technologies Private Limited, Banagalore (IN)

(21) Appl. No.: 16/766,446

(22) PCT Filed: May 25, 2018
(86) PCT No.: PCT/IB2018/053733

§ 371 Date: May 22, 2020

(30) Foreign Application Priority Data
Nov. 23, 2017 (IN) 201741041950

(51) Int. Cl. A61B 6/00; A61B 6/03

(52) U.S. Cl. CPC A61B 6/4085; A61B 6/505; A61B 6/482; A61B 6/466; A61B 6/032

(57) ABSTRACT

The present invention relates to a system and method to visualize both soft-tissue and hard bone anatomy of an object. For this purpose, the system (100) of the present invention comprises a ring gantry (101) with two x-ray sources (102, 103) to simultaneously project radiations of different intensities to capture both soft-tissue and hard bone anatomy of the object. Further x-ray detectors (104, 105) are placed opposite to the x-ray sources (102, 103) to capture image data pertaining to both soft-tissue and hard bone anatomy of the object. A computer (106) to receive image data from the detectors and to generate single CBCT volume which has the information of both soft tissue and their relative hard-bone anatomy captured within the same time interval so as to identify any relative displacement of the soft tissue with respective to their hard bone anatomy in the generated volume.

### CROSS-REFERENCE TO RELATED APPLICATIONS

The present application is national phase and claims priority to PCT application serial no. PCT/IB2018/053733, filed May 25, 2018, which claims priority to Indian patent application serial no. 201741041950, filed Nov. 23, 2017, all herein incorporated by reference in their entireties.

### DESCRIPTION OF THE INVENTION

### Technical Field of the Invention

The present invention relates to medical imaging systems and more particularly relates to a system and method to visualize both soft-tissue and hard bone anatomy of an object using a medical imaging system.

### BACKGROUND OF THE INVENTION

In radiotherapy, Image-guided radiotherapy (IGRT) plays a major role in locating the tumor and aligning it with respect to the therapy beam. An example of IGRT would include a cone beam computed tomography (CBCT) in which the object is irradiated by cone beam type X-irradiation from several different imaging angles to produce CT-type (Computed Tomography) imaging data of the object being imaged.

In general, the CBCT system comprises an X-ray source to generate radiation required for imaging directed from at least one imaging angle at the object being imaged and an X-ray detector for receiving the radiation that has passed through the object for producing the image data. The CBCT system also comprises a computer operationally coupled to the X-ray source and X-ray detector to receive image data of an object and to facilitate real-time volume build up and real-time visualization of the object.

Further, an important factor in the delivery of image guided radiation therapy is the quality of image used to plan and deliver the radiation therapy, and particularly the accuracy with which each anatomical structure such as a soft tissue with respect to the corresponding hard bone anatomy is identified so as to enable the operator to locate the area of interest and align it with respect to the therapy beam.

However, in the existing IGRT system if the parameters are set to identify the soft tissue then the hard bone anatomy with respect to the soft tissue is not captured within the same interval of time. Hence, there exists a need to capture both soft tissue and hard bone anatomy concurrently within the same time interval so as to clearly identify the soft tissues and their respective positions with respect to the hard bone anatomy thereby enabling the operator to locate the area of interest more accurately and to align it with respect to the therapy beam.

For instance, the US patent document U.S. Pat. No. 6,898,263B2 (referred herein as '263) discloses about a system and method for soft-tissue volume visualization. For this purpose, the method comprises the steps of scanning an object using a multi-energy computed tomography (MECT) system to obtain image data for an object. Here, the anatomic image data so obtained includes a high-energy image and a low-energy image. The anatomic image data is then decomposed to obtain a first density image representing soft-tissue within the object and a second density image representing bone material within the object. The high energy image, low-energy image, soft-tissue density image and bone material density image are then segmented to obtain a soft-tissue image including bone anatomy for the region of interest within the anatomy.

However, the MECT system as disclosed in '263 is configured to acquire projections sequentially at different x-ray tube potentials so as to generate a first density image representative of bone material and a second density image representative of soft-tissue.

Hence, there exists a need to capture both soft tissue and hard bone anatomy concurrently within the same time interval so as to clearly identify the soft tissues and their respective positions with respect to the hard bone anatomy thereby enabling the operator to locate the area of interest more accurately and align it with respect to the therapy beam.

### SUMMARY OF THE INVENTION

The present invention overcomes the drawbacks of the prior art by providing a system and method that uses dual-energy imaging technique to visualize both soft-tissue and hard bone anatomy of an object. For this purpose, the system of the present invention comprises a ring gantry with two x-ray sources to simultaneously project radiations of different intensities to capture both soft-tissue and hard bone anatomy of the object. Here, detectors are placed opposite to the x-ray sources to capture image data pertaining to both soft-tissue and hard bone anatomy of the object. Further, a computer is operationally coupled to the radiation sources and radiation detectors to receive image data pertaining to both soft-tissue and hard bone anatomy of the object and to generate a single three-dimensional CBCT volume which has the information of both soft tissue and their relative hard-bone anatomy captured simultaneously by the x-ray detectors so as to identify any relative displacement of the soft tissue with respective to their hard bone anatomy in the generated volume.

In accordance to one embodiment of the present invention, the computer upon receiving the image data is configured to generate a first three-dimensional cone beam computed tomography (CBCT) volume pertaining to the received hard-bone anatomy of the object and a second three-dimensional CBCT volume pertaining to the received soft-tissue anatomy of the object. The computer then combines the first three dimensional CBCT volume pertaining to the hard-bone anatomy of the object and the second CBCT volume pertaining to the soft-tissue anatomy of the object to form a single CBCT volume which has the information of both soft tissue and their relative hard-bone anatomy captured simultaneously by the x-ray detectors.

In accordance to one embodiment of the present invention, a method to visualize both soft-tissue and hard bone anatomy of an object using a dual-energy cone beam computed tomography (DECBCT) technique comprises the steps of projecting radiations of varying intensities simultaneously by two x-ray sources to capture image data pertaining to both soft-tissue and hard bone anatomy of the object within the same interval of time. The method collects the image data pertaining to both soft-tissue and hard bone anatomy of the object generated by detectors by using a computer. Here, the computer upon receiving the image data generates a single cone beam computed tomography (CBCT) volume which has the information of both soft-tissue and their relative hard bone anatomy captured simultaneously by the x-ray detectors to identify any relative displacement inside the generated volume.

Thus, the system and method of the present invention uses dual-energy imaging technique to generate a single cone beam computed tomography (CBCT) volume which has the information of both soft tissue and their relative hard-bone anatomy captured simultaneously within the same interval of time to identify any relative displacement inside the volume thereby enabling the operator to locate the area of interest more accurately and to align it with respect to the therapy beam.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates a perspective view of a dual energy cone beam computed tomography (DECBCT) system to visualize both soft-tissue and hard bone anatomy of an object, in accordance to one or more embodiment of the present invention.

FIG. 2 is a flow chart representing a method to visualize both soft-tissue and hard bone anatomy of an object using a dual energy cone beam computed tomography (DECBCT) technique, in accordance to one or more embodiment of the present invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

While the present invention has been described with respect to certain embodiments, it will be apparent to those skilled in the art that various changes and modification may be made without departing from the scope of the invention as defined in the following claims.

The methods and systems described herein apply dual-energy imaging technique to volume visualization. Techniques that allow visualization of three-dimensional data are referred to as volume rendering. Here, the system of the present invention comprises a ring gantry with two x-ray sources to simultaneously project radiations of varying intensities to capture both soft-tissue and hard bone anatomy of the object and a plurality of x-ray detectors placed opposite to the x-ray sources to capture image data pertaining to both soft-tissue and hard bone anatomy of the object. The system also comprises a computer to receive the image data pertaining to both soft-tissue and hard bone anatomy of the object from the detectors, so as to generate a single cone beam computed tomography (CBCT) volume which has the information of both soft tissue and their relative hard-bone anatomy.

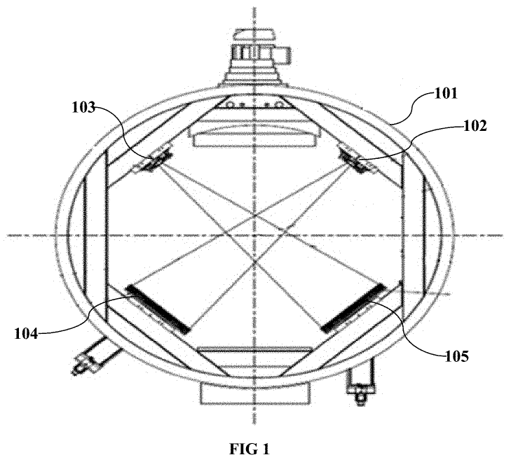

FIG. 1 illustrates a perspective view of a dual energy cone beam computed tomography (DECBCT) system to visualize both soft-tissue and hard bone anatomy of an object, in accordance to one or more embodiment of the present invention.

As shown in FIG. 1, the dual energy cone beam computed tomography (DECBCT) system (100) mainly comprises a ring gantry (101) that has two x-ray sources (102, 103) to simultaneously project radiations of different intensities to capture both soft-tissue and hard bone anatomy of the object. Further, two x-ray detectors (104, 105) are placed opposite to the two x-ray sources (102, 103) to capture image data pertaining to both soft-tissue and hard bone anatomy of the object. Further, a computer (106) is operationally coupled to the radiation sources i.e. x-ray sources (102, 103) and radiation detectors i.e. x-ray detectors (104, 105) to receive image data pertaining to both soft-tissue and hard bone anatomy. The computer (106) upon receiving the image data generates a first three-dimensional cone beam computed tomography (CBCT) volume pertaining to the received hard-bone anatomy of the object and a second three-dimensional CBCT volume pertaining to the received soft-tissue of the object.

The computer (106) then combines the generated two CBCT volumes to generate a single CBCT volume which has the information of both soft tissue and their relative hard-bone anatomy to identify any relative displacement inside the volume so as to enable the operator to locate the area of interest more accurately and align it with respect to the therapy beam. Thus, during a scan to acquire projection data, gantry (101) and the components mounted on the gantry rotate about a center of rotation.

The DECBCT system (100) also has a patient positioning system to align the located area of interest with respect to the target beam.

In accordance to one embodiment of the present invention, the x-ray sources (102, 103) further comprises a first x-ray source (102) and a second x-ray source (103). Here, the first x-ray source (102) is configured to project radiations of high-energy beam to capture hard bone anatomy of the object namely a patient, and a second x-ray source (103) to project radiations of low-energy beam to capture soft-tissue anatomy of the object. This could be interchanged as per configuration. For illustrative purpose, let us consider that the first x-ray source operates at 160 kilo volt (kVp) potentials to project radiations of high-energy beam and the second x-ray source operates at 80 kVp to project radiations of low-energy beam.

In accordance to one embodiment of the present invention, the x-ray detectors (104, 105) further comprises a first x-ray detector (104) and a second x-ray detector (105). Here, the first x-ray detector (104) is placed opposite to the first x-ray source (102) to capture image data pertaining to the hard bone anatomy of the object. Similarly, the second x-ray detector (105) is placed opposite to the second x-ray source (103) to capture image data pertaining to the soft-tissue anatomy of the object.

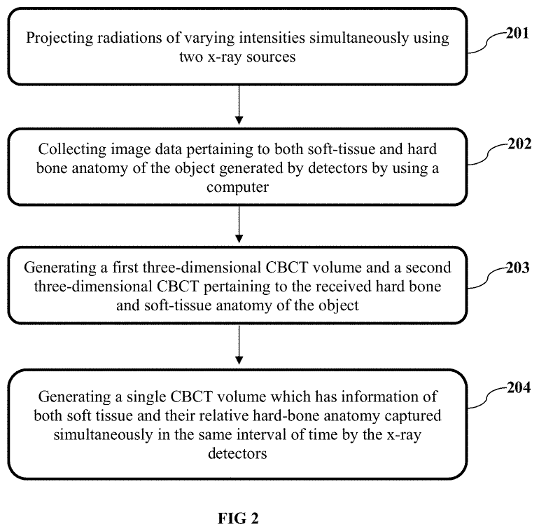

FIG. 2 is a flow chart representing a method to visualize both soft-tissue and hard bone anatomy of an object using a dual energy cone beam computed tomography (DECBCT) technique, in accordance to one or more embodiment of the present invention. As shown in FIG. 2, radiations of different intensities are projected simultaneously by both the x-ray sources to capture image data pertaining to both soft-tissue and hard bone anatomy of the object within the same interval of time at step 201. At step 202, computer collects the acquired projection data generated by detectors to receives the image data pertaining to both soft-tissue and hard bone anatomy of the object.

At step 203, the computer generates a first three-dimensional cone beam computed tomography (CBCT) volume pertaining to the received hard-bone anatomy of the object and a second three-dimensional CBCT volume pertaining to the received soft-tissue anatomy of the object. The computer then combines the generated first three dimensional CBCT volume pertaining to the hard-bone anatomy of the object and the second CBCT volume pertaining to the soft-tissue of the object to form a single CBCT volume at step 204 which has the information of both soft tissue and their relative hard-bone anatomy captured simultaneously by the x-ray detectors so as to identify any relative displacement inside the volume thereby enabling the operator to locate the area of interest more accurately and align it with respect to the therapy beam.

Thus, the system and method of the present invention uses dual-energy imaging technique to generate a single cone beam computed tomography (CBCT) volume which has the information of both soft tissue and their relative hard-bone anatomy captured simultaneously by the x-ray detectors to identify any relative displacement inside the volume so as to enable the operator to locate the area of interest more accurately and to align it with respect to the therapy beam.

While the present invention has been described with respect to certain embodiments, it will be apparent to those skilled in the art that various changes and modification may be made without departing from the scope of the invention as defined in the following claims.

We claim:

1. A dual energy cone beam computed tomography (DECBCT) system (100) comprises:
a) a ring gantry (101) with two x-ray sources (102, 103) to simultaneously project radiations of varying intensities to capture both soft-tissue and hard bone anatomy of an object;
b) x-ray detectors (104, 105) placed opposite to the x-ray sources (102, 103) to capture image data pertaining to both soft-tissue and hard bone anatomy of the object;
c) a computer (106) operationally coupled to the radiation sources i.e. x-ray sources (102, 103) and radiation detectors i.e. x-ray detectors (104, 105) to receive image data pertaining to both soft-tissue and hard bone anatomy of the object, wherein the computer upon receiving the image data is configured to:
generate a first three-dimensional cone beam computed tomography (CBCT) volume pertaining to the received hard-bone anatomy of the object and a second three-dimensional CBCT volume pertaining to the received soft-tissue anatomy of the object;
combine the generated first three-dimensional CBCT volume pertaining to the hard-bone anatomy of the object and the second CBCT volume pertaining to the soft tissue anatomy of the object to form a single CBCT volume which has the information of both soft tissue and their relative hard-bone anatomy captured simultaneously by the x-ray detectors so as to identify any relative displacement of the soft tissue with respective to their hard bone anatomy in the generated volume.

2. The DECBCT system (100) as claimed in claim 1, wherein the x-ray sources (102, 103) further comprises:
a) a first x-ray source (102) to project radiations of high-energy beam to capture hard bone anatomy of the object; and
b) a second x-ray source (103) to project radiations of low-energy beam to capture soft-tissue anatomy of the object.

3. The DECBCT system (100) as claimed in claim 2, wherein the x-ray detector (104, 105) further comprises:
a) a first x-ray detector (104) placed opposite to the first x-ray source (102) to capture image data pertaining to the hard bone anatomy of the object; and
b) a second x-ray detector (105) placed opposite to the second x-ray source (103) to capture image data pertaining to the soft-tissue anatomy of the object.

4. A method to visualize both soft-tissue and hard bone anatomy of an object using a dual energy cone beam computed tomography (DECBCT) technique, comprising the steps of:
a) projecting radiations of dual intensities simultaneously by at least one x-ray source to capture image data pertaining to both soft-tissue and hard bone anatomy of the object within a same interval of time;
b) collecting the acquired image data pertaining to both soft-tissue and hard bone anatomy of the object generated by detectors by using a computer, wherein the computer upon receiving the image data generates a single cone beam computed tomography (CBCT) volume which has the information of both soft tissue and their relative hard-bone anatomy captured simultaneously so as to identify any relative displacement inside the volume thereby enabling the operator to locate the area of interest more accurately and align it with respect to the therapy beam.

5. The method as claimed in claim 4, wherein the method for generating a single cone beam computed tomography (CBCT) volume, comprising the steps of:
a) generating a first three-dimensional cone beam computed tomography (CBCT) volume pertaining to the received hard-bone anatomy of the object;
b) generating a second three-dimensional CBCT volume pertaining to the received soft-tissue anatomy of the object; and
c) combining the first three-dimensional CBCT volume pertaining to the hard-bone anatomy of the object and the second CBCT volume pertaining to the soft-tissue anatomy of the object to form a single CBCT volume which has the information of both soft tissue and their relative hard-bone anatomy captured simultaneously by the x-ray detectors to identify any relative displacement inside the volume thereby enabling the operator to locate the area of interest more accurately and align it with respect to the therapy beam.
